(19) United States
(12) Patent Application Publication
(10) Pub. No.: US 2011/0038909 A1
(43) Pub. Date: Feb. 17, 2011

(54) System and Method for Using Nanoparticles for Antimicrobial Activity

(75) Inventors: Paul ROE, Elkhart, IN (US); Michael JOBE, Elkhart, IN (US); Michael NOVICK, Miami, FL (US)

Correspondence Address: AKERMAN SENTERFITT, P.O. BOX 3188, WEST PALM BEACH, FL, 33402-3188, US

(73) Assignee: Heirloom Holdings, Miami, FL (US)

(21) Appl. No.: 12/901,265

(22) Filed: Oct. 8, 2010

Related U.S. Application Data
(63) Continuation-in-part of application No. 11/562,554, filed on Nov. 22, 2006.

(51) Int. Cl. A01N 25/08; A01N 59/16; B05D 5/00; B05D 1/02; B05D 1/18; A01P 1/00; B82Y 5/00

(52) U.S. Cl. 424/405; 424/618; 427/256; 427/427.4; 427/430.1; 977/773

(57) ABSTRACT

A system and method for using nanoparticles for antimicrobial activity. An antimicrobial coating can be provided, which can include a plurality of discrete first particles that can have an average diameter of less than approximately 400 nanometers and, in a preferred embodiment, 10 nanometers or less. The plurality of discrete first particles can include pure silver metal. In an embodiment, the plurality of discrete first particles can also include a physiologically inert stabilizer. The antimicrobial coating can also include a polymeric film, in which the plurality of discrete first particles can be directly embedded therein.

### CROSS-REFERENCE TO RELATED APPLICATION

This patent application is a continuation-in-part application to U.S. patent application Ser. No. 11/562,554, filed Nov. 22, 2006, the entirety of which is incorporated herein by reference.

### STATEMENT REGARDING FEDERALLY SPONSORED RESEARCH OR DEVELOPMENT

Not applicable.

### FIELD OF THE INVENTION

The present invention relates to antimicrobial coatings, and, more particularly, to antimicrobial coatings that may be applied to upholstery.

### BACKGROUND OF THE INVENTION

For years, the upholstery industry has produced materials that either had no antimicrobial activity or provided an antimicrobial activity using environmentally objectionable compounds such as arsenic and cyanide. While many polymers used to make exposed surfaces in the upholstery industry are recycleable, recycling of plastics containing arsenic and cyanide is limited by environmental regulations, such as the Resource Conservation and Recovery Act (RCRA). Thus, both pre-consumer and post-consumer recycling of plastics used in upholstery is limited when either arsenic or cyanide are used to provide antimicrobial activity.

Some metals, such as silver, gold, platinum, palladium, iridium, copper, tin, antimony, bismuth, and zinc, are known to be effective antimicrobials. The antimicrobial activity of these metals is commonly referred to as the oligodynamic effect.

Antimicrobial metals are believed to function by releasing metal ions that are absorbed by microbes coming into contact with the metal ions. The metal ions react with proteins in the microbe to render the protein insoluble and thereby biologically inactive. The inactive proteins perturb cellular function, disrupt membranes, and prevent the normal activity and reproduction of DNA. The precipitation of proteins and subsequent effects caused by the precipitation ultimately kills the microbe. This mechanism may be limited by, the fact that it requires the microbe to directly contact the metal or a fluid containing ions of the metal.

Of the metals mentioned above, silver in the form of silver metal (Ag) or silver oxide (Ag₂O) is perhaps the best known oligodynamic metal due to its unusually good antimicrobial activity at low concentrations. However, silver is a precious metal that is relatively expensive and is known to turn an opaque-black in the presence of oxygen. In the past, these properties have limited the use of silver as an antimicrobial in upholstery applications. In general, the silver at, or near, the surface provides the bulk of the oligodynamic activity because the microbe must generally contact the silver directly. Thus, the use of silver throughout an upholstery material is neither a cost-effective option nor provides the desired antimicrobial activity. In addition, because both silver and silver oxide are black, neither has been seen as an option for use in a clear coating applied to a material so that the vivid colors or patterns of an underlying material are visible.

It has proven difficult to create an antimicrobial polymeric coating or film using metal particles. Typically, additives, such as active ingredients and fillers, are compounded with thermoplastic polymers and the resulting plastic is produced in a pelletized form. These pellets are then used in various material forming processes, including extrusion, injection molding, compression molding, etc. As with other additives, in the final materials formed using the compounded pellets, the metal particles are generally buried beneath the surface. Unlike many of the active ingredients in final materials formed from pellets, post-formation treatments cannot be used to make metal fibers bloom to the surface. Since the antimicrobial activity of the oligodynamic metals requires direct interaction between the metal and the microbe, the metals buried beneath the surface impart the coated material with little or no antimicrobial activity.

Another area of interest in the upholstery industry is materials exhibiting resistance to ultraviolet (UV) radiation. Plastics are generally degraded by exposure to UV radiation. This is of particular concern for upholstery exposed for prolonged periods to direct sunlight, such as upholstery used on boats. As with antimicrobial agents, there is a desire to localize the UV inhibitors to where they are useful and to apply the UV inhibitor without covering the colors or patterns of the underlying material.

### SUMMARY OF THE INVENTION

The present invention is drawn to thin polymer coatings having a high antimicrobial activity. Within 30 minutes of exposure, these coatings are capable of killing up to 99.9% of the bacteria that contact them. Coatings of the present invention have shown a 99.9% efficacy against bacteria of the genera Staphylococcus and Enterococcus. The coatings are also capable of killing and inhibiting viruses, parasites, and fungi, including the fungi responsible for mold and mildew. This efficacy is the result of accessible nanoparticles that include oligodynamic metal that are directly supported by the polymer coating.

An embodiment of the invention relates to an antimicrobial coating which may include a polymeric film and a plurality of discrete first particles that have diameter of about 400 nanometers or less. The discrete first particles may include silver metal, silver oxide, or a combination of both. The discrete first particles may be in direct contact with the polymeric film. In addition to the discrete first particles, the coating may also include more than one second particles that include zinc oxide.

The second particles may have an average particle size of less than about 1 micrometer. Preferably, the second particles may have an average particle size of about 400 nanometers or less. The second particles may preferably have an average particle size of about 100 nanometers or less, and more preferably about 80 nanometers or less.

The discrete first particles may have an average diameter of less than about 1 micrometer. The discrete first particles may have an average diameter of about 400 nanometers or less, preferably about 100 nanometers or less. In some arrangements, the discrete first particles may have an average diameter of about 50 nanometers or less, more preferably less than about 20 nanometers, and most preferably less than about 10 nanometers. In some arrangements, the preferred diameter of the discrete first particles ranges from about 2 to about 8 nanometers, preferably from about 4 to about 6 nanometers.

The discrete first particles may constitute about 8 wt-% or less of the coating. The discrete first particles may constitute about 0.2 wt-% or less of the coating.

The coating may have an overall thickness of about 50 micrometers or less. The overall thickness of the coating may be about 5 micrometers or less. The coating may be clear. More specifically, the coating may be transparent. The polymeric film may include a polymer including, but not limited to a vinyl, an acrylic, a polyester, a polyurethane, a melamine, an epoxy, a polycarbonate, and a polyolefin.

Another embodiment of the present invention is an article with an antimicrobial surface. The article includes a surface with an antimicrobial coating deposited thereon. The coating includes a plurality of discrete first particles having an average diameter of about 400 nanometers or less that are supported directly by a polymeric film. The discrete first particles comprise at least one of silver metal, silver oxide, or a combination thereof. The discrete first particles may have a surface area to mass ratio of about 1.4 square meters per gram or greater.

The discrete first particles may have an average diameter of less than about 1 micrometer. The discrete first particles may have an average diameter of about 400 nanometers or less or even about 100 nanometers or less. In some arrangements, the discrete first particles may have an average diameter of about 50 nanometers or less, more preferably less than about 20 nanometers, and most preferably less than about 10 nanometers. In some arrangements, the preferred diameter of the discrete first particles ranges from about 2 to about 8 nanometers, preferably from about 4 to about 6 nanometers.

The discrete first particles may constitute about 8 wt-% or less of the coating. The discrete first particles may constitute about 0.2 wt-% or less of the coating.

The coating may have a thickness of approximately 50 micrometers or less. The coating may have a thickness of approximately 5 micrometers or less. The coating may be clear. Specifically, the coating may be transparent. The polymeric film may include a polymer including, but not limited to, a vinyl, an acrylic, a polyester, a polyurethane, a melamine, an epoxy, a polycarbonate, and a polyolefin.

The coating may include more a plurality of second particles that may include zinc oxide. The second particles may have an average particle size of less than about 1 micrometer. The second particles may have an average particle size of about 400 nanometers or less. The second particles may preferably have an average particle size of about 100 nanometers or less, and more preferably about 80 nanometers or less.

Another embodiment of the present invention is a method of imparting an antimicrobial activity to a surface of a material. The method may include blending a solution used to form a coating with an antimicrobial activity. The solution may include a solvent, a polymeric resin, and a plurality of discrete first particles that may have an average diameter of about 400 nanometers or less. The discrete first particles may include at least one of silver metal, silver oxide, and a combination thereof. The solution may be applied to a surface portion of a material. After applying the solution of the surface, the solution on the surface of the material may be dried. The discrete first particles may have a surface area to mass ratio of about 1.4 square meters per gram or greater

The discrete first particles may have an average diameter of about 400 nanometers or less, preferably about 100 nanometers or less. In some arrangements, the discrete first particles may have an average diameter of about 50 nanometers or less, more preferably less than about 20 nanometers, and most preferably less than about 10 nanometers. In some arrangements, the preferred diameter of the discrete first particles ranges from about 2 to about 8 nanometers, preferably from about 4 to about 6 nanometers.

The solution may include the discrete first particles in an amount of about 2 wt-% or less or even as low as about 0.01 wt-% or less. Alternatively, the solution may include the discrete first particles in an amount between about 0.01 wt-% and about 2 wt-%. The polymeric resin may constitute less than or equal to about 25 wt-% of the solution.

The thickness of the coating as applied may be less than about 250 micrometers. The thickness of the coating as applied may be less than about 100 micrometers, more preferably less than about 50 micrometers. The coating may be clear. Specifically, the coating may be transparent.

The solution may be applied to the surface of the material using a coating apparatus including, but not limited to, a gravure coating apparatus, a spray coating apparatus, and a flood coating apparatus. The coating apparatus used to apply the solution may be a gravure coating apparatus.

The polymeric resin in the solution may be a polymer including, but not limited to, a vinyl, an acrylic, a polyester, a polyurethane, a melamine; an epoxy, and a polycarbonate. The solvent in the solution may be a fluid including, but not limited to, water, methyl ethyl ketone, methyl isobutyl ketone, acetone, toluene, ethyl acetate, methyl acetate, propyl acetate, n-methyl-2-pyrrolidone, tetrahydrofuran, glycol and glycol ethers.

The solution may include a plurality of second particles that include zinc oxide. The second particles may have an average particle size of less than about 1 micrometer. Preferably, the second particles may have an average particle size of about 400 nanometers or less. The second particles may preferably have an average particle size of about 100 nanometers or less, and more preferably about 80 nanometers or less.

Another embodiment of the present invention is directed to an antimicrobial coating, which can include a plurality of discrete first particles that can have an average diameter of less than approximately 400 nanometers. The plurality of discrete first particles can include pure silver metal. In an embodiment, the plurality of discrete first particles can also include a physiologically inert stabilizer. The antimicrobial coating can also include a polymeric film, in which the plurality of discrete first particles can be directly embedded therein.

Yet another embodiment of the present invention can include an article, which can include an antimicrobial surface. An antimicrobial coating can be bonded to a surface of the article. The coating can include a plurality of discrete first particle having an average diameter of approximately 400 nanometers or less. Notably, the plurality of discrete first particles can include pure silver and a stabilizer. The antimicrobial coating can also include a polymeric film, in which the plurality of discrete first particles can be directly embedded therein.

In still another embodiment, the present invention can include a method of imparting an antimicrobial activity to a surface of a material. The method can include providing a solution for forming a coating with an antimicrobial activity. The solution can include a plurality of discrete first particles having an average diameter of approximately 400 nanometers or less and the plurality of discrete first particles can include pure silver and a stabilizer. Additionally, the solution can include a solvent and a polymeric resin. The method can further include applying the solution to a surface portion of the material.

An advantage of the inventive coatings is that they exhibit an extremely high antimicrobial activity using low concentration of oligodynamic metals. Another advantage of the present invention is that the antimicrobial activity of the coating is not transient because the oligodynamic particles are supported directly by the coating. Thus, a material with the inventive antimicrobial coating deposited thereon may be washed with conventional cleaners without washing away the antimicrobial activity of the coating.

Another advantage of the present invention is that while the oligodynamic particles are directly supported by the film, a large percentage of the particles remain at the surface to provide a coating having a high oligodynamic activity. Thus, the present invention provides coatings having a high oligodynamic activity using a small quantity of silver and without using a separate support structure, such as a zeolite, for the silver.

Another advantage of the present invention is that it is feasible to apply the coating possessing antimicrobial and ultraviolet inhibiting properties directly onto materials with vivid colors and patterns. Because antimicrobial coatings of the present invention may be transparent or translucent, the antimicrobial coatings may be applied directly to existing fabrics that have vivid colors or patterns without damaging the aesthetic appeal of the colors or patterns. Thus, rather than including the expensive antimicrobial particles, ultraviolet inhibiting particles, or both, throughout the entire material a manufacturer may localize these active ingredients in an antimicrobial surface coating. In addition, a retailer may easily apply the inventive coating to add antimicrobial properties, ultraviolet inhibiting properties, or both, to materials selected by a customer.

Another advantage of the present invention is that it provides antimicrobial activity with improved conformity with government regulations compared to previous antimicrobial formulations used in the upholstery industry. Previous antimicrobial upholstery formulations relying on arsenic or cyanide failed to comply with government recycling regulations, for example, RCRA.

These and other embodiments are described in more detail below.

### BRIEF DESCRIPTION OF THE DRAWINGS

The features of the invention, which are believed to be novel, are set forth with particularity in the appended claims. The embodiments herein, can be understood by reference to the following description, taken in conjunction with the accompanying drawings, in the several figures of which like reference numerals identify like elements, and in which:

FIG. 1 is a cross-sectional view of a material coated with a coating that includes a polymeric film and discrete first particles.

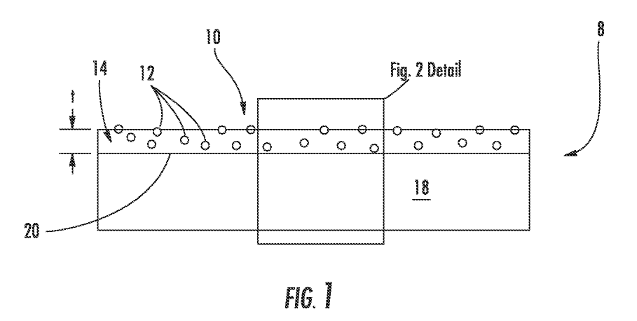

FIG. 2 is a detailed view of the coated material of FIG. 1, showing well dispersed discrete first particles that are smaller than the wavelength of impinging visible light.

FIG. 3 is a detailed view of the coated material of FIG. 1, showing tightly packed antimicrobial discrete first particles that are smaller than the wavelength of impinging visible light.

FIG. 4 is a cross-sectional view of a material coated with a coating that includes a polymeric film, antimicrobial discrete first particles, and ultraviolet radiation inhibiting second particles.

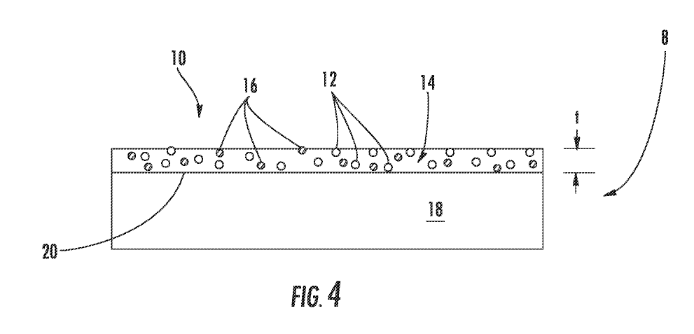

FIG. 5 is a schematic of a gravure coating apparatus that includes an impression roller to force the material web against the gravure roll with sufficient force to transfer the solution to the material.

FIG. 6 is a schematic of a gravure coating apparatus that relies on tension rollers to press the material web against the surface of the gravure roll with sufficient force to transfer the solution to the material.

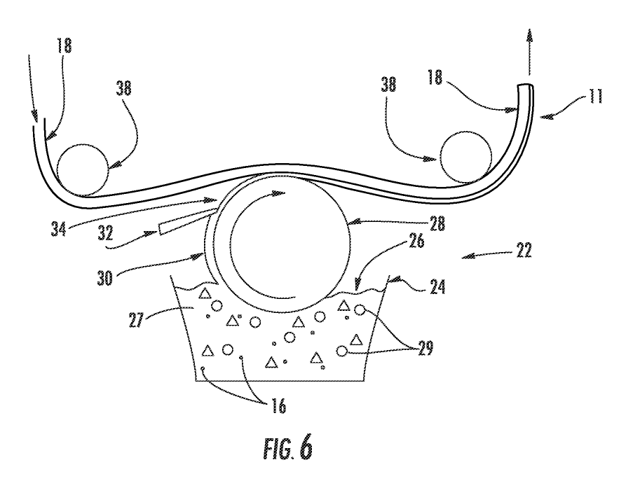

### DETAILED DESCRIPTION

The present invention is directed to antimicrobial coatings that may be applied to materials used for upholstery. In one embodiment, the invention is directed to an antimicrobial coating 10 that includes a polymeric film 14 and more than one discrete first particles 12 having an average diameter of about 400 nanometers or less. The discrete first particles 12 may include at least one of silver metal, silver oxide, or a combination thereof. The discrete first particles 12 may be directly supported by the polymeric film 14. The coating may generally be applied to the surface 20 of an article 18 to form an antimicrobial material 8. The discrete first particles 12 may have a surface area to mass ratio of approximately 1.4 square meters per gram or greater.

As used herein, “antimicrobial” has its common meaning and indicates that a substance kills or inhibits the growth of microbes such as bacteria, fungi, viruses, or parasites. As used herein, “particle” has its normal meaning and also includes fibers. As used herein, “diameter” generally has its common meaning; however, the length of the fiber is the diameter of a particle that is a fiber. As described herein, “discrete” generally refers to a particle that is not supported by a carrier material, for instance a zeolite or other similar materials, other than the polymer film of the present invention. As used herein, “clear” is used to describe an antimicrobial coating that is transparent or transluscent.

As used herein, “silver” and “silver metal” refer to materials containing at least 50 wt-% or more silver (Ag) content. In some cases, “silver” may refer to materials containing 80 wt-% or more Ag, in yet other cases “silver” may refer to materials with at least 90 wt-%, 92.5 wt-%, 95 wt-%, or even 99.9 wt-% Ag.

As used herein, a material “directly supported” by a film indicates that no carrier other than the film is used to support the material. For example, a film containing silver metal or silver ions supported by a separate support structure, for example a zeolite, would not contain silver directly supported by the film.

The antimicrobial coating 10 may be clear. In addition, the antimicrobial coating 10 may be transparent. The discrete first particles 12 may have an average diameter of less than about 1 micrometer (1000 nanometers). The discrete first particles 12 may be even smaller and have an average diameter of less than about 100 nanometers. In some arrangements, the discrete first particles 12 may have an average diameter of about 50 nanometers or less, more preferably less than about 20 nanometers, and most preferably less than about 10 nanometers. In some arrangements, the preferred diameter of the discrete first particles 12 ranges from about 2 to about 8 nanometers, preferably from about 4 to about 6 nanometers.

The antimicrobial coating 10 may also contain one or more second particles 16 that include an ultraviolet (UV) radiation inhibitor, such as zinc oxide. The second particles 16 may have an average diameter of less than 1 micrometer. The second particles 16 may have an average particle size of about 400 nanometers or less. The second particles 16 may preferably have an average particle size of about 100 nanometers or less, and more preferably about 80 nanometers or less.

Visible light is the name given to the portion of the electromagnetic spectrum visible to the human eye. The visible light seen by most people has a wavelength between about 400 nanometers and about 700 nanometers. In order of decreasing wavelength, the visible light spectrum includes red, orange, yellow, green, blue, and violet. Violet light has the shortest wavelength, between about 380-450 nanometers, and red light has the longest wavelength, between about 620-750 nanometers. The wavelengths of the other colors present in the visible light spectrum are shown below:

| | |
|---|---|
| Violet | 380-450 nm |
| Blue | 450-495 nm |
| Green | 495-570 nm |
| Yellow | 570-590 nm |
| Orange | 590-620 nm |
| Red | 620-750 nm |

Ultraviolet (UV) radiation is broken up into three categories: UV A having a wavelength ranging from about 315 nanometers to about 400 nanometers, UV B having a wavelength ranging from about 280 nanometers to about 315 nanometers, and UV C having a wavelength ranging from about 100 nanometers to about 280 nanometers. Although all three will damage upholstery, only UV A and UV B are generally present at the Earth's surface because Earth's atmosphere reflects a significant amount of the UV B spectrum and nearly all of the UV C spectrum back into space.

In some embodiments of the present invention, the antimicrobial coating 10 may be clear. The antimicrobial coating 10 will appear to be clear when the impinging visible light 40 is not reflected by the polymeric film 14 or any particles 12, 16 in the antimicrobial coating 10. A clear antimicrobial coating 10 is possible when the polymeric film 14 is transparent or translucent and the particles 12, 16 present in the antimicrobial coating 10 are smaller than the wavelength (λ) of visible light and well dispersed in the coating 10. For the coating 10 to appear to be clear, the particles 12, 16 must be small enough and spaced out enough that a significant amount of the electromagnetic waves that make up visible light pass through the antimicrobial coating 10 without striking any particles 12, 16.

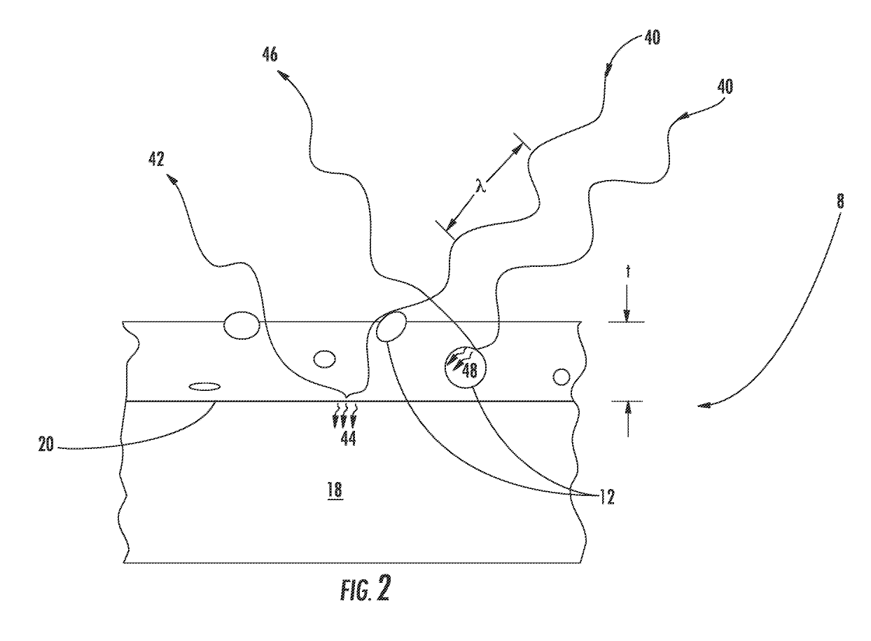

FIG. 2 shows a diagram of an antimicrobial material with a transparent polymeric film and a plurality of discrete first particles 12. In this arrangement, impinging visible light 40 will strike either the surface 20 of the underlying material 18 or one of the discrete first particles 12. If the impinging visible light 40 strikes the surface 20 of the underlying material 18, some of the impinging visible light 40 will be absorbed 44 by the surface 20 while the remainder will be reflected 42 by the surface 20. If the impinging visible light 40 strikes a first particle 12, some of the impinging visible light 40 will be absorbed 48 by the first particle 12 while the remainder will be reflected 46 by the first particle 12. A person observing an antimicrobial coating 10 where a substantial amount of the impinging visible light 40 is reflected 46 by the first particles 12 will not be able to see the colors and patterns of the surface 20 underlying the coating 10. However, an antimicrobial coating 10 will appear clear and the colors and patterns of the underlying surface 20 will be apparent to a person observing a coating 10 where a substantial amount of the impinging visible light 40 is reflected 42 by the surface 20.

If the polymeric film 14 is transparent or translucent and the particles 12, 16 directly supported in the polymeric film 14 are dispersed and smaller than the wavelength (λ) of the impinging visible light 40, a significant portion of the impinging visible light 40 will be reflected 42 by the underlying surface 20 of the material 18.

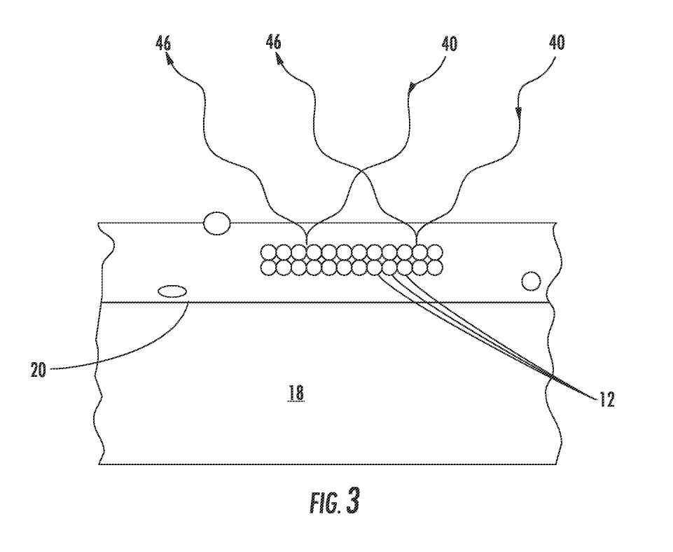

As shown in FIG. 3, if the particles are not dispersed, most, if not all, of the impinging visible light 40 may be reflected 46 or absorbed 48 by the first particles 12 and the antimicrobial coating 10 will appear opaque and exhibit the color of the first particles 12. As can be seen, this is true even if the first particles 12 are much smaller than the wavelength of the impinging visible light 40.

In order to make the antimicrobial coating 10 transparent, the particles 12, 16 incorporated into the coating 10 will generally be smaller than the wavelength (λ) of all the colors of visible light. The lower end of the visible light spectrum observed by most people, 400 nanometers, is within the violet portion of the visible light spectrum. Thus, a polymeric film 14 with dispersed particles 12, 16 having an average diameter of about 400 nanometers or less may be transparent for all colors. Similarly, well dispersed particles 12, 16 having an average diameter of about 100 nanometers or less would be transparent for all colors. In some arrangements, the discrete first particles 12 may have an average diameter of about 50 nanometers or less, more preferably less than about 20 nanometers, and most preferably less than about 10 nanometers. In some arrangements, the preferred diameter of the discrete first particles 12 ranges from about 2 to about 8 nanometers, preferably from about 4 to about 6 nanometers.

Using the knowledge set forth above, an antimicrobial coating 10 that is clear when applied to a red surface 20 may be designed using particles 12, 16 having an average diameter greater than 400 nanometers. Red light has a wavelength between about 620 and about 750 nanometers. Thus, red wavelengths could pass through a transparent polymeric film 14 that includes well dispersed particles 12, 16 having a diameter of about 620 nanometers or less. Accordingly, an antimicrobial coating 10 containing well dispersed particles 12, 16 with a diameter of about 620 nanometers or less may appear clear when applied to a red surface 20.

Based on the foregoing, one skilled in the art may create other embodiments that appear clear though they contain particles 12, 16 having an average diameter of up to about 620 nanometers. For example, particles 12, 16 having an average diameter of about 590 nanometers or less for orange and red surfaces 20, and particles 12, 16 having an average diameter of about 495 nanometers or less for green, yellow, orange and red surfaces 20.

In addition to the antimicrobial properties of the present invention, it is possible to include a plurality of second particles 16 to provide protection against UV radiation. A second particle 16 useful in the present invention may include zinc oxide. Zinc oxide inhibits UV degradation by reflecting nearly all impinging radiation that strikes the particle. Based on this mechanism, in order to inhibit UV degradation of the surface 20, the second particles 16 must be large enough that they exceed the wavelength of the impinging UV radiation they are designed to protect against. Thus, to provide a clear antimicrobial coating 10 having UV inhibiting properties, the second particles 16 would generally have an average diameter larger than about 350 nanometers, but small enough that the colors of the underlying surface 20 would be visible using the techniques discussed above. In order to protect against all UV A, the second particles 16 may have an average diameter of about 400 nanometers or greater. In order to protect against all UV B radiation, the second particles 16 may have an average diameter of about 315 nanometers or greater.

The theoretical discussion herein relates to particles 12, 16 placed perfectly between the peaks of the impinging visible radiation 40. In actual practice, the particles 12, 16 are not perfectly spaced. Accordingly, if actual practice particles 12, 16 may need to have a diameter significantly smaller than 400 nanometers in order for the coating 10 to appear to be clear. Similarly, particles 12, 16 smaller than 350 nanometers may be useful to protect the underlying surface 20 of a material 18 from impinging UV radiation.

The ability to provide a transparent antimicrobial coating 10 has several benefits. First, it allows a manufacturer of materials to produce a single base material that does not include either an antimicrobial activity or ultraviolet inhibiting properties. This may improve manufacturing efficiency by reducing the number of different codes, or materials, that need to be manufactured on a base machine. Upon request, the manufacturer can apply a thin coating 10 with an antimicrobial activity, ultraviolet inhibiting properties, or both, to the prefabricated material 18, rather than the entire material 8. In addition, this means a smaller amount of these expensive additives is necessary to provide at least equivalent protection. Second, an aftermarket designer or retailer may easily apply the inventive coating 10 to add antimicrobial properties, ultraviolet inhibiting properties, or both, to prefabricated materials 18. This provides a broader selection of material for both the aftermarket designer and the customer.

The antimicrobial coating 10 may include discrete first particles 12 in an amount of about 8 wt-% or less. In another arrangement, the antimicrobial coating 10 may include discrete first particles 12 in an amount of about 0.2 wt-% or less. Studies have shown that up to 99.9% of bacteria of the genera Staphylococcus and Enterococcus are killed after 30 minutes of exposure to the inventive antimicrobial coatings 10 having silver or silver oxide particles 12 present at a concentration of about 8 wt-% or less. The same 99.9% efficacy has been shown with inventive coatings containing silver or silver oxide particles 12 at a concentration of about 0.2 wt-% or less.

The antimicrobial coatings 10 of the present invention may have a thickness (t) of about 50 microns or less. In another embodiment, the coatings may have a thickness (t) of approximately 5 microns or less.

The polymeric film 14 of the present invention may include a polymer including, but not limited to, a vinyl, an acrylic, a polyester, a polyurethane, a melamine, an epoxy, a polycarbonate, and a polyolefin.

The coating of the present invention may be applied to the surface 20 of an upholstery material 20. The upholstery material 20 may be any material appropriate for the particular application. In some embodiments, the antimicrobial coating 10 may be applied to any number of upholstery materials 20 including, but not limited to, supported or unsupported vinyls, polyurethanes, olefins, and leathers.

The invention also includes a method of imparting an antimicrobial activity to a surface 20 of a material 18. The method includes making a solution 26 that includes a solvent 27, a polymeric resin 29, and a plurality of discrete first particles 12 with an average diameter of about 400 nanometers or less. The discrete first particles 12 may include silver metal, silver oxide, or a combination thereof. The solution 26 is applied to the surface 20 of a material 18. The solution 26 may be dried after it is applied to the surface 20. The discrete first particles 12 may have a surface area to mass ratio of approximately 1.4 square meters per gram or greater.

The solution 26 may also include one or more second particles 16 that include zinc oxide. The second particles 16 may have an average particle size of less than about 1 micrometer. The discrete first particles 12 may be even smaller and have an average diameter of less than about 100 nanometers. In some arrangements, the discrete first particles 12 may have an average diameter of about 50 nanometers or less, more preferably less than about 20 nanometers, and most preferably less than about 10 nanometers. In some arrangements, the preferred diameter of the discrete first particles 12 ranges from about 2 to about 8 nanometers, preferably from about 4 to about 6 nanometers.

The polymeric resin 29 may amount to about 25 wt-% or less of the solution. The polymeric resin 29 may be a polymer including, but not limited to, a vinyl, an acrylic, a polyester, a polyurethane, a melamine, an epoxy, and a polycarbonate. In general, the polymers of the present invention are obtained in the form of waterborne or solvent-based resin systems.

The solvent 27 may be a fluid including, but not limited to, water, methyl ethyl ketone, methyl isobutyl ketone, acetone, toluene, ethyl acetate, methyl acetate, propyl acetate, n-methyl-2-pyrrolidone, tetrahydrofuran, glycol, glycol ethers, and combinations thereof. Specific glycols that may be useful in the present invention include ethylene glycol. Specific glycol ethers that may be useful in the present invention include ethylene glycol monobutyl ether, which is sold under the trade name Butyl Cellusolve.

The antimicrobial coating 10 as applied in the inventive method may have a thickness of about 250 micrometers or less. In the alternative, the antimicrobial coating 10 as applied in the inventive method may have a thickness of about 25 micrometers or less. Because the wet weight of the antimicrobial coating 10 may only include about 25 wt-% or less of polymer, the dried coatings may have a thickness that is approximately 20-25% of the thickness of the coating as applied.

The solution 26 may be applied using an apparatus including but not limited to a gravure coating apparatus 22, a spray coating apparatus, and a flood coating apparatus. In one arrangement, a gravure coating apparatus 22 may be used to apply the solution to the surface 20 of the material 18.

The viscosity of the solution 26 applied using a gravure roll coating apparatus 22 is an important variable that should be controlled for an effective, high-efficiency coating operation. Since the amount of polymeric resin 29 in solution 26 may impact the antimicrobial and UV inhibiting properties of the resulting coating 10, the solution 26 in the reservoir 24 may be heated or cooled to adjust the viscosity as desired. The viscosity of the solution 26 may also be modified by adding more or less of various ingredients, including, but not limited to, polymeric resins 29, solvents 27, surfactants, defoaming agents, and other additives.

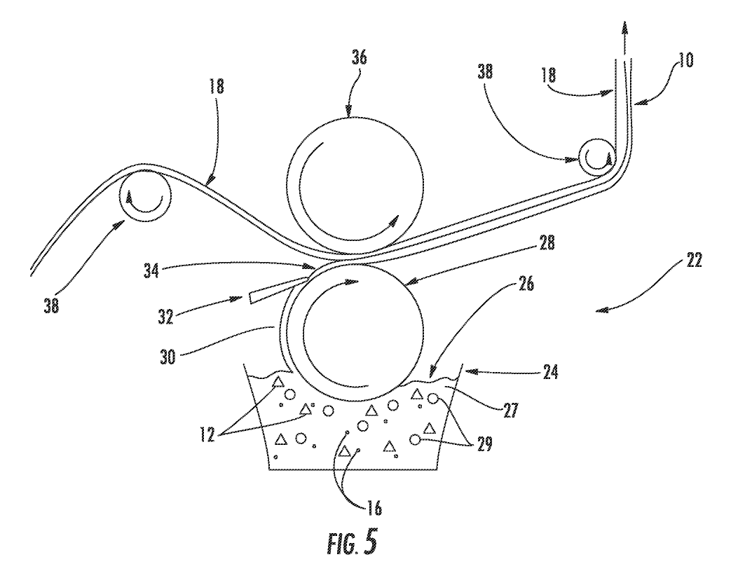

FIGS. 5 & 6 show schematics of gravure coating apparatuses 22 that may be useful in the present invention. In gravure coating, a gravure roll 28 includes a number of small openings on its surface, and rotates in contact with both a reservoir 24 of solution 26 and the material 18 being coated. The small surface openings take up the solution 26 from the reservoir 24 and transfer it to the material 18. In many gravure coating apparatuses 22, a thick film 30 of solution 26 coats the gravure roll 28 on the portion emerging from the reservoir 24. A doctor blade 32 can be used to wipe excess solution 26 from the surface of the gravure roll 28. By this mechanism, a thinner film 34 of solution 26 can be applied to the material 18. Thus, the depth and size of the small surface openings in the gravure roll 28 can determine the amount of solution 26 that is transferred to the surface 20 of the material 18 to be coated.

The gravure roll coating apparatus generally relies on tension rollers 38 to maintain tension on the material 18 web being coated. As shown in FIG. 5, the gravure roll coating apparatus 22 may also include one or more pressure rollers 36 that directly force the web material 18 down onto the gravure roll 28.

In one flood coating arrangement, a gravure roll coating apparatus 22 may also be used without a doctor blade 32. Generally, this is possible where the viscosity of the solution 26 is adjusted so that the desired amount of solution 26 is applied without the need of a doctor blade 32. The uniformity, temperature, and viscosity of the solution 26 are factors that may be used to determine the amount of solution 26 applied and ensure uniformity in both the machine direction and the cross direction.

Spray coating may also be a useful method for applying the solution 26 of the present invention. Many process parameters should be controlled to ensure uniform consistency of spray coatings. Relevant process parameters include, but are not limited to, temperature, humidity, fluid pressure, fluid viscosity and uniformity of the solution. For instance, failure to control humidity may cause undesirable results such as blushing and surface skinning of the antimicrobial coating 10.

Surprisingly, the antimicrobial coatings 10 of the present invention exhibit superior antimicrobial activity compared to coatings made from polymer compounded with the same weight percent of discrete oligodynamic metal particles 12. This indicates that the inventive coatings 10 may have a larger percentage of discrete antimicrobial particles 12 at the surface of the antimicrobial coating 10 than would be present using polymer compounded with the discrete antimicrobial particles 12. It is believed this results because the particles 12, 16 are suspended in the solvent 27 and are deposited on the surface of the polymeric resin 29 particles when the solvent 27 evaporates. Similarly, the inventive coatings may have a larger percentage of UV inhibiting particles 16 at the surface of the coating 10 than would be present using polymer compounded with the UV inhibiting particles 16.

In an embodiment, the coatings described above can be configured to include silver-based nanopowders, which can be utilized as antimicrobial agents. For example, the coatings described in the present disclosure can include one or more forms of SmartSilver™, which are manufactured and available through NanoHorizons, Inc. of State College, Pa. In an embodiment, the coatings can include SmartSilver™ AS (EPA Registration #83507-2), which can include pure silver (up to 99.99%) and a physiologically inert stabilizer. SmartSilver™ AS is a dispersible silver nanopowder that can be utilized as an antimicrobial agent to hinder and/or kill various types of microbes. The actual silver content for the dispersible nanopowder can be 51.9% w/w and the recommended loading level for dry and finished goods can be 0.0385-0.385% w/w (0.02-0.2% Ag w/w). In an embodiment, the silver nanopowder can be configured to tolerate a pH range ranging from 3 to 11 (6.5-9.5 in deionized water) and a moisture content level of less than 1%. In another embodiment, the silver nanopowder can have a dark green appearance.

The dispersible silver nanopowder can be soluble in a wide range of solvents such as, but not limited to, water (at temperatures less than or equal to 40° C.), chloroform, diethylene glycol, dimethyl formamide, ethanol, isopropanol (95%), methanol, propylene glycol, and tetrahydrofuran. In an embodiment, the solution generated by dispersing the silver nanopowder in the above solvents can have an opaque green appearance. In order to facilitate dispersion in various types of solvents, several methods can be utilized. For example, in order to facilitate dispersion of the silver-based nanopowder in water according to a preferred method, the silver-based nanopowder can be slowly added to agitated 50-60° C. water in order to generate a high-solids slurry/solution.

In an embodiment, the silver nanopowder can be insoluble in water at temperatures greater than 40° C. Considering the insolubility of the nanopowder at 40° C., the adding of the silver nanopowder to the agitated 50-60° C. water can cause the nanopowder to hydrate slowly and also prevent clumping. After mixing the silver nanopowder with the agitated water, cold water can be added to the solution to reach a preferred concentration level. The mixing can continue until the silver nanopowder is effectively dissolved in the water. In another embodiment, the silver nanopowder can be added slowly to well-agitated cold water, such as room temperature water that is less than or equal to 35° C. In this embodiment, slowly adding the silver nanopowder to the water can prevent the powder from sticking and clumping, and thereby decrease the amount of time needed to dissolve the nanopowder. With regard to the other solvents described above, the silver nanopowder can be added slowly to the solvent and heat can be applied to help the nanopowder dissolve more rapidly. Additionally, the nanopowder can be utilized in bandages, textile finishes, sprays, and paints.

Another form of SmartSilver™ that can be utilized in the coatings can be SmartSilver™ WS (EPA Registration #83507-3). SmartSilver™ WS is a polymer-stabilized silver nanopowder that can be utilized as an antimicrobial agent in water soluble applications that can appear as a dark brown powder. The nanopowder can be soluble in water at 1 g/ml. When mixed with water to form a solution, SmartSilver™ WS can have an opaque brown appearance and can appear as clear yellow at dilute concentrations. In an embodiment, the silver content can be 32.5% w/w and the recommended loading level for dry and finished goods can be 0.0615-0.615% w/w (0.02-0.2% Ag w/w). Much like for SmartSilver™ AS, the stabilized nanopowder can be configured to tolerate a pH range ranging from 3 to 11 (6.5-9.5 in deionized water) and a moisture content level of less than 1%. In addition to the SmartSilver™ varieties discussed in the present disclosure, other forms of SmartSilver™ can be utilized as well.

In one embodiment according to the present invention, an antimicrobial coating can be provided. The antimicrobial coating can include a plurality of discrete first particles, which can have an average diameter of less than approximately 400 nanometers. In a preferred embodiment, the average diameter can be less than or equal to 10 nanometers, which can allow for the particles to cover a larger surface area. Notably, the discrete first particles can include silver metal and a physiologically inert stabilizer. The silver can be pure silver and/or the silver found in various forms of SmartSilver™, such as found in the forms described above. The physiologically inert stabilizer can be a polymer or polymer variant. For example, the polymer can include polyurethane, melamine, epoxy, polycarbonate and/or the polymers and stabilizers used in SmartSilver™ variants. Additionally, the plurality of discrete first particles do not have to be supported by a carrier material such as a zeolite. The antimicrobial coating can also include a polymeric film, in which, the plurality of discrete first particles can be directly embedded. The polymeric film can include vinyl, an acrylic, a polyester, a polyurethane, a melamine, an epoxy, a polycarbonate, a polyolefin, and other types of polymers.

The antimicrobial coating can be applied to upholstery and/or other similar materials to kill and prevent microbial growth. Bacteria and fungus, which are often transmitted to the surface of upholstery by moisture, can cause various health-related problems and odors. However, the moisture which carries the bacteria and/or fungus can be utilized to activate the silver particles in the antimicrobial coating. By activating the silver particles, it allows for the release of antimicrobial silver ions. The silver ions can be released at a stable/controlled rate and can then bind to the proteins of the bacteria and/or fungus and deactivate the bacteria and/or fungus. Once deactivated, odors can be removed and the bacteria and/or fungus can be killed. This increases the durability of the upholstery, while having no negative impact on the physical integrity of the upholstery.

In an embodiment, the antimicrobial coating can further include a plurality of second particles. The plurality of second particles can include zinc oxide or other similar substances, which can be utilized to inhibit UV degradation by reflecting nearly all impinging radiation that strikes the particle. Notably, the plurality of second particles can have average particle sizes of 400 nanometers or less. In another embodiment, the antimicrobial coating can be transparent and have a clear appearance so as to not block the color of the upholstery.

In one embodiment, an article including an antimicrobial surface can be provided. The article can include an antimicrobial coating, which can be bonded to one or more surfaces of the article. Notably, the antimicrobial coating can include a plurality of discrete first particles, which can have an average diameter of approximately 400 nanometers or less. In a preferred embodiment, the first particles have an average diameter of 10 nanometers or less. The first particles can include pure silver (up to 99.99%) and a stabilizer. The stabilizer can be a physiologically inert stabilizer such as the stabilizers described according to the present disclosure. The pure silver and/or the stabilizer can be directly embedded in a polymeric film. Polymeric film can include polymers such as a vinyl, an acrylic, a polyester, a polyurethane, a melamine, an epoxy, a polycarbonate, and a polyolefin.

In still another embodiment, the present disclosure can include a method of imparting an antimicrobial activity to a surface of a material. Initially, the method can include providing a solution for forming a coating with an antimicrobial activity. The solution can include a plurality of discrete first particles, which can have an average diameter of approximately 400 nanometers or less and, in a preferred embodiment, 10 nanometers or less. Notably, the plurality of first particles can include pure silver and/or stabilizers. Additionally, the solution can include a solvent. Solvents can include, but are not limited to including, chloroform, diethylene glycol, dimethyl formamide, ethanol, isopropanol (95%), methanol, propylene glycol, tetrahydrofuran, along with other solvents such as those described above. The solution can further include a polymeric resin, which can include, but is not limited to including, a vinyl, an acrylic, a polyester, a polyurethane, a melamine, an epoxy, and a polycarbonate. Finally, the method can also include applying the solution to the surface portion of the material.

The foregoing is provided for purposes of illustrating, explaining, and describing embodiments of this invention. Modifications and adaptations to these embodiments will be apparent to those skilled in the art and may be made without departing from the scope or spirit of this invention. The arrangements described herein are intended to provide a general understanding of the structure of various embodiments, and they are not intended to serve as a complete description of all the elements and features of apparatus and systems that might make use of the structures described herein. Many other arrangements will be apparent to those of skill in the art upon reviewing the above description. Other arrangements may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Thus, although specific arrangements have been illustrated and described herein, it should be appreciated that any arrangement calculated to achieve the same purpose may be substituted for the specific arrangement shown. This disclosure is intended to cover any and all adaptations or variations of various embodiments and arrangements of the invention. Combinations of the above arrangements, and other arrangements not specifically described herein, will be apparent to those of skill in the art upon reviewing the above description. Therefore, it is intended that the disclosure not be limited to the particular arrangement(s) disclosed as the best mode contemplated for carrying out this invention, but that the invention will include all embodiments and arrangements falling within the scope of the appended claims.

We claim:

1. An antimicrobial coating comprising:
a plurality of discrete first particles having an average diameter of less than approximately 400 nanometers, wherein the plurality of discrete first particles comprises pure silver metal; and
a polymeric film, wherein the plurality of discrete first particles are directly embedded in the polymeric film.

2. The antimicrobial coating of claim 1, wherein the plurality of discrete first particles further comprise a physiologically inert stabilizer.

3. The antimicrobial coating of claim 1, wherein the silver metal comprises approximately one of 32.5% and 51.9% of the antimicrobial coating by weight.

4. The antimicrobial coating of claim 1, wherein the discrete first particles have an average diameter of 10 nanometers or less.

5. The antimicrobial coating of claim 1, wherein the antimicrobial coating is formed by a solution comprising a solvent mixed with the plurality of first particles.

6. The antimicrobial coating of claim 5, wherein the solvent comprises at least one of water, chloroform, diethylene glycol, dimethyl formamide, ethanol, isopropanol, methanol, propylene glycol, and tetrahydrofuran.

7. The antimicrobial coating of claim 1, wherein the plurality of first particles are configured to tolerate a pH range from 3 to 11.

8. The antimicrobial coating of claim 1, wherein the antimicrobial coating is transparent.

9. The antimicrobial coating of claim 1, wherein the coating has a thickness of approximately 50 microns or less.

10. The antimicrobial coating of claim 1, wherein the polymeric film comprises a polymer selected from the group consisting of a vinyl, an acrylic, a polyester, a polyurethane, a melamine, an epoxy, a polycarbonate, and a polyolefin.

11. The antimicrobial coating of claim 1, further comprising a plurality of second particles, wherein the plurality of second particles comprise zinc oxide.

12. The antimicrobial coating of claim 9, wherein the second particles have an average diameter of approximately 400 nanometers or less.

13. An article with an antimicrobial surface, comprising:
a surface of the article; and
an antimicrobial coating bonded to said surface, wherein said coating comprises,
a plurality of discrete first particles having an average diameter of approximately 400 nanometers or less, wherein the plurality of discrete first particles comprise pure silver and a stabilizer, and
a polymeric film, wherein the plurality of discrete first particles are directly embedded in the polymeric film.

14. The article of claim 13, wherein the article is a material selected from the group consisting of supported or unsupported vinyls, polyurethanes, olefins, and leathers.

15. The article of claim 13, wherein the discrete first particles have an average diameter of 10 nanometers or less.

16. The article of claim 13, wherein the silver metal comprises approximately one of 32.5% and 51.9% of the antimicrobial coating by weight.

17. The article of claim 13, wherein stabilizer is physiologically inert.

18. The article of claim 13, further comprising a plurality of second particles, wherein the plurality of second particles comprise zinc oxide.

19. The article of claim 18, wherein the plurality of second particles prevent UV degradation of the article by reflecting nearly all impinging radiation that strikes the plurality of second particles.

20. A method of imparting an antimicrobial activity to a surface of a material, comprising:
providing a solution for forming a coating with an antimicrobial activity, said solution comprising,
a plurality of discrete first particles having an average diameter of approximately 400 nanometers or less, wherein the plurality of discrete first particles comprise pure silver and a stabilizer, and
a solvent, and
a polymeric resin; and
applying the solution to a surface portion of the material.

21. The method of claim 20, wherein the material is selected from the group consisting of supported or unsupported vinyls, polyurethanes, olefins, and leathers.

22. The method of claim 20, wherein the moisture content of at least one of the plurality of first particles and the coating is less than 1%.

23. The method of claim 20, wherein the plurality of discrete first particles comprise between approximately 0.01% and approximately 2% by weight of the solution.

24. The method of claim 20, wherein the polymeric resin comprises less than or equal to approximately 25% by weight of the solution.

25. The method of claim 20, wherein the applying step further comprises
using an apparatus for applying the solution to the surface of the material, wherein the apparatus is selected from the group consisting of a gravure coating apparatus, a spray coating apparatus, and a flood coating apparatus.

26. The method of claim 25, wherein the apparatus used to apply the solution is a gravure coating apparatus.

27. The method of claim 20, wherein the solvent comprises one of water, chloroform, diethylene glycol, dimethyl formamide, ethanol, isopropanol, methanol, propylene glycol, methyl ethyl ketone, methyl isobutyl ketone, acetone, toluene, ethyl acetate, methyl acetate, propyl acetate, n-methyl-2-pyrrolidone, tetrahydrofuran, glycol and glycol ethers.

28. The method of claim 20, wherein the solution further comprises a plurality of second particles, wherein the plurality of second particles comprise zinc oxide, and wherein the plurality of second particles prevent UV degradation of the material by reflecting nearly all impinging radiation that strikes the plurality of second particles.
